(19) United States
(12) Patent Application Publication
(10) Pub. No.: US 2023/0024119 A1
(43) Pub. Date: Jan. 26, 2023

(54) COMBINED USE OF SODIUM TRANS-[TETRACHLORIDOBIS(1H-INDAZOLE)RUTHENATE(III)] AND ETOMOXIR FOR TREATING CANCERS

(71) Applicant: Bold Therapeutics, Inc., Vancouver (CA)

(72) Inventors: Dina BAIER, Vancouver (CA); Beatrix SCHOENHACKER-ALTE, Vancouver (CA); Christopher GERNER, Vancouver (CA); Petra HEFFETER, Vancouver (CA); Bernhard KEPPLER, Vancouver (CA); Walter BERGER, Vancouver (CA)

(21) Appl. No.: 17/782,867

(22) PCT Filed: Dec. 4, 2020
(86) PCT No.: PCT/CA2020/051674

§ 371 Date: Jun. 6, 2022

Related U.S. Application Data
(60) Provisional application No. 62/944,318, filed on Dec. 5, 2019.

(51) Int. Cl. A61K 31/555; A61K 31/336

(52) U.S. Cl. CPC A61K 31/555; A61K 31/336

(57) ABSTRACT

Methods are provided for treating a cancer in a human patient in need thereof, comprising administering an effective amount of sodium trans-[tetrachloridobis(1H-indazole)ruthenate(III)] and a effective amount of etomoxir.

### FIELD

The invention is in the field of therapeutic compounds, particularly the combined use of sodium trans-[tetrachloridobis(1H-indazole)ruthenate(III)] and etomoxir for treating cancers.

### BACKGROUND

Sodium trans-[tetrachloridobis(1H-indazole)ruthenate(III)] is a coordinated complex of ruthenium having anticancer activity (also known as KP1339, NKP-1339, IT-139, and Na[RuIIICl4(Hind)2]). Methods of making alkali metal salts of trans-[tetrachlorobis(1H-indazole)ruthenate (III)] are for example described in PCT Patent Application No. PCT/US2018/031436, such compounds having Formula I:

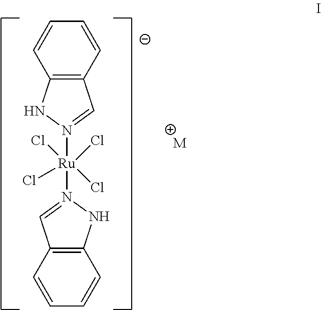

wherein M is an alkali metal cation, including the sodium salt:

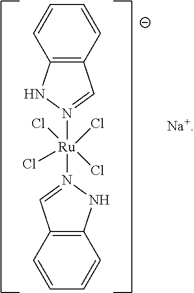

Etomoxir or 2[6(4-chlorophenoxy)hexyl]oxirane-2-carboxylate, is an inhibitor of carnitine palmitoyltransferase-1 (CPT-1), an activity which inhibits the formation of acyl carnitines, a metabolic step that is involved in the transport of fatty acyl chains from the cytosol into the intermembrane space of mitochondria. Etomoxir has also been described as an agonist of PPARα, and in clinical use as enhancing feelings of hunger.

Acquired therapy resistance of diverse cancer types frequently involves changed metabolic processes linked to altered cellular lipid uptake or de novo synthesis.

### SUMMARY

Methods are provided for treating a cancer in a human patient in need thereof, comprising administering an effective amount of sodium trans-[tetrachloridobis(1H-indazole)ruthenate(III)] and a effective amount of etomoxir. The effective amount of sodium trans-[tetrachloridobis(1H-indazole)ruthenate(III)] and the effective amount of etomoxir may for example be synergistically effective for treating the cancer. The sodium trans-[tetrachloridobis(1H-indazole)ruthenate(III)] and the etomoxir may for example be for sequential administration, in any order, or for combined administration in a co-formulation.

The cancer to be treated may for example be a cancer that is resistant to treatment with sodium trans-[tetrachloridobis(1H-indazole)ruthenate(III)] alone, and may for example be a colon cancer or a pancreatic cancer.

### DETAILED DESCRIPTION

Disclosed herein is an unexpected impact of lipid metabolism in resistance against the anticancer ruthenium compound sodium trans-[tetrachloridobis(1H-indazole)ruthenate(III)] (also referred to herein as KP1339 or BOLD-100 or IT139). Also disclosed is a combination therapy using KP1339 and a lipid metabolism modulator, including etomoxir and analogues thereof, for enhanced activity and resistance prevention. The effectiveness of this combination is evidenced in colon and pancreatic cancer cells, selected for resistance to KP1339. In particular, HCT116 and Capan1 cell models with acquired KP1339 resistance were established and investigated for altered gene expression profiles. mRNA data were confirmed on the protein level using immunoblotting. Lipid storage compartments (lipid droplets) were visualized fluorescently with Bodipy 493/503. Utilizing specific pharmacological inhibitors, the role of altered lipid metabolism components in KP1339 resistance was dissected. Mitochondrial oxygen consumption rate (OCR) and extracellular acidification rate (ECAR) were assessed by Seahorse XF analysis. Lipidomics and consecutive proteomics analysis were used to further characterize the resistance phenotype.

Resistance in both cell models was not based on a drug accumulation defect. In-depth bioinformatic analyses revealed major changes in the lipid metabolism associated with KP1339 resistance. Consistently, KP1339-resistant cells contained elevated lipid droplet levels. Hence, resistant cell models were hypersensitive towards lipid-modulating agents triacsin C, statins, and orlistat. Unexpectedly, the β-oxidation inhibitor etomoxir massively synergized with KP1339 and completely reverted acquired resistance. In the Seahorse Mito Stress Test, KP1339 massively reduced ECAR in sensitive HCT116 cells suggesting interference with glycolysis. Accordingly, KP1339-resistant cells exhibited clearly reduced spontaneous ECAR levels which, in contrast to the sensitive parental line, did not increase upon respiration inhibition by oligomycin. Upon KP1339 treatment, OCR was reduced in parental but stayed unchanged in resistant cells. Lipidomics confirmed distinctly enhanced lipid droplet component levels (e.g. triglycerides) associated with KP1339 resistance while proteomics indicated degradation of monocarboxylate transporters MCT-1/MCT-4.

Additional embodiments of the present invention provide methods for preparing drug products containing the sodium salt of trans-[tetrachlorobis(1H-indazole)ruthenate (III)] (i.e. KP1339 or KT-139 or BOLD-100).

One aspect of the current invention provides a method for preparing a sterile, lyophilized drug product containing sodium trans-[tetrachlorobis(1H-indazole)ruthenate (III)]. This formulation would be suitable for administration to a patient. The formulation is comprised of sodium trans-[tetrachlorobis(1H-indazole)ruthenate (III)], a pH buffer, and a cryoprotective agent. The general method for providing said formulation comprises the steps of preparing aqueous buffer solution, preparing aqueous cryoprotectant solution, dissolution of sodium trans-[tetrachlorobis(1H-indazole)ruthenate (III)] in the buffer solution, addition of the cryoprotectant solution, sterile filtration (e.g. aseptic filtration), filling of vials under sterile conditions, and lyophilization under sterile conditions. Suitable buffers include, but are not limited to: citrate, TRIS, acetate, EDTA, HEPES, tricine, and imidazole. The use of a phosphate buffer is possible but is not preferred. A preferred aspect of the present invention is the use of a citric acid/sodium citrate buffer. Suitable cryoprotective agents include, but are not limited to: sugars, monosaccarides, disaccharides, polyalcohols, mannitol, sorbitol, sucrose, trehalose, dextran, and dextrose. A preferred aspect of the present invention is the use of mannitol as the cyroprotecive agent.

As described above, herein, sodium trans-[tetrachlorobis(1H-indazole)ruthenate (III)] can degrade in water to Compound A (Scheme II). One skilled in the art will recognize that limiting this degradation reaction would be advantageous to obtaining the highest purity product. It was found that cooling the sodium trans-[tetrachlorobis(1H-indazole)ruthenate (III)] solution during the formulation process was found to greatly reduce the amount of Compound A present in the lyophilized product. In one aspect of the invention, the sodium trans-[tetrachlorobis(1H-indazole)ruthenate (III)] solution is cooled to 4° C. during the formulation process. In another aspect of the invention, the sodium trans-[tetrachlorobis(1H-indazole)ruthenate (III)] solution is cooled to 2-8° C. during the formulation process. In another aspect of the invention, the sodium trans-[tetrachlorobis(1H-indazole)ruthenate (III)] solution is cooled to 2-15° C. during the formulation process.

One embodiment of the present invention provides a composition comprising sodium trans-[tetrachlorobis(1H-indazole)ruthenate (III)], a suitable buffer, and mannitol. In some embodiments, a suitable buffer comprises a citrate buffer. For instance, in some embodiments, a citrate buffer comprises sodium citrate and citric acid.

One embodiment of the present invention provides a composition comprising sodium trans-[tetrachlorobis(1H-indazole)ruthenate (III)], sodium citrate, citric acid, and mannitol.

One embodiment of the present invention provides a composition comprising sodium trans-[tetrachlorobis(1H-indazole)ruthenate (III)], sodium citrate, citric acid, mannitol, and mer,trans-[RuIIICl3(Hind)2(H2O)].

One embodiment of the present invention provides a composition comprising sodium trans-[tetrachlorobis(1H-indazole)ruthenate (III)], sodium citrate, citric acid, mannitol, mer,trans-[RuIIICl3(Hind)2(H2O)], and a cesium salt.

One embodiment of the present invention provides a composition comprising sodium trans-[tetrachlorobis(1H-indazole)ruthenate (III)], sodium citrate, citric acid, and mannitol, wherein the sodium trans-[tetrachlorobis(1H-indazole)ruthenate (III)] is amorphous.

One embodiment of the present invention provides a composition comprising sodium trans-[tetrachlorobis(1H-indazole)ruthenate (III)], sodium citrate, citric acid, mannitol, and mer,trans-[RuIIICl3(Hind)2(H2O)], wherein the sodium trans-[tetrachlorobis(1H-indazole)ruthenate (III)] is amorphous.

One embodiment of the present invention provides a composition comprising sodium trans-[tetrachlorobis(1H-indazole)ruthenate (III)], sodium citrate, citric acid, mannitol, mer,trans-[RuIIICl3(Hind)2(H2O)], and a cesium salt, wherein the sodium trans-[tetrachlorobis(1H-indazole)ruthenate (III)] is amorphous.

One embodiment of the present invention provides a composition comprising sodium trans-[tetrachlorobis(1H-indazole)ruthenate (III)], sodium citrate, citric acid, mannitol, mer,trans-[RuIIICl3(Hind)2(H2O)], and a cesium salt;

wherein:

mer,trans-[Ru^{III}Cl₃(Hind)₂(H₂O)] is between about 0.01 and about 0.4 weight percent of the composition,

and cesium is between about 0.00001 and about 0.01 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

mer,trans-[Ru^{III}Cl₃(Hind)₂(H₂O)] is between about 0.01 and about 0.4 weight percent of the composition,

and cesium is between about 0.00001 and about 0.01 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

mer,trans-[Ru^{III}Cl₃(Hind)₂(H₂O)] is between about 0.01 and about 0.2 weight percent of the composition,

and cesium is between about 0.00001 and about 0.01 weight percent of the composition.

One embodiment of the present invention provides a composition comprising sodium trans-[tetrachlorobis(1H-indazole)ruthenate (III)], mer,trans-[Ru^{III}Cl₃(Hind)₂(H₂O)], and a cesium salt;

wherein:

mer,trans-[Ru^{III}Cl₃(Hind)₂(H₂O)] is between about 0.01 and about 0.40 weight percent of the composition,

and cesium is between about 0.00001 and about 0.01 weight percent of the composition.

One embodiment of the present invention provides a composition comprising sodium trans-[tetrachlorobis(1H-indazole)ruthenate (III)], mer,trans-[Ru^{III}Cl₃(Hind)₂(H₂O)], and a cesium salt;

wherein:

the composition is a lyophilized powder,

mer,trans-[Ru^{III}Cl₃(Hind)₂(H₂O)] is between about 0.01 and about 0.40 weight percent of the composition,

and cesium is between about 0.00001 and about 0.01 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

the composition is a lyophilized powder,

mer,trans-[Ru^{III}Cl₃(Hind)₂(H₂O)] is between about 0.01 and about 0.3 weight percent of the composition,

and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

mer,trans-[Ru^{III}Cl₃(Hind)₂(H₂O)] is between about 0.01 and about 0.3 weight percent of the composition,

and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

the composition is a lyophilized powder,

sodium trans-[tetrachlorobis(1H-indazole)ruthenate (III)] is about 11.5 to about 14.0 weight percent of the composition,

citric acid is about 43.9 to about 53.7 weight percent of the composition,

sodium citrate is about 25.7 to about 23.1 weight percent of the composition,

mannitol is about 11.5 to about 14.0 weight percent of the composition,

mer,trans-[Ru^{III}Cl₃(Hind)₂(H₂O)] is about 0.01 and about 0.3 weight percent of the composition,

and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

the composition is a lyophilized powder,

sodium trans-[tetrachlorobis(1H-indazole)ruthenate (III)] is about 10.2 to about 15.3 weight percent of the composition,

citric acid is about 39.0 to about 58.5 weight percent of the composition,

sodium citrate is about 20.5 to about 30.8 weight percent of the composition,

mannitol is about 10.2 to about 15.3 weight percent of the composition,

mer,trans-[Ru^{III}Cl₃(Hind)₂(H₂O)] is about 0.01 and about 0.3 weight percent of the composition,

and cesium is between about 0.00001 and about 0.1 weight percent of the composition.

One embodiment of the present invention provides a composition comprising sodium trans-[tetrachlorobis(1H-indazole)ruthenate (III)], sodium citrate, citric acid, mannitol, mer,trans-[Ru^{III}Cl₃(Hind)₂(H₂O)], and a cesium salt;

wherein:

the composition is a lyophilized powder,

sodium trans-[tetrachlorobis(1H-indazole)ruthenate (III)] is about 10.2 to about 15.3 weight percent of the composition,

mer,trans-[Ru^{III}Cl₃(Hind)₂(H₂O)] is about 0.01 and about 0.3 weight percent composition,

and cesium is between about 0.00001 and about 0.1 weight percent of the composition.

One embodiment of the present invention provides a composition comprising sodium trans-[tetrachlorobis(1H-indazole)ruthenate (III)], mannitol, citric acid, and sodium citrate;

wherein:

sodium trans-[tetrachlorobis(1H-indazole)ruthenate (III)] is about 49.86 weight percent of the composition,

mannitol is about 49.86 weight percent of the composition,

citric acid is about 0.187 weight percent of the composition,

and sodium citrate is about 0.093 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, and sodium citrate;

wherein:

sodium trans-[tetrachlorobis(1H-indazole)ruthenate (III)] is about 40 to about 60 weight percent of the composition,

mannitol is about 40 to about 60 weight percent of the composition,

citric acid is about 0.01 to about 0.5 weight percent of the composition,

and sodium citrate is about 0.001 to about 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, and sodium citrate;

wherein:

sodium trans-[tetrachlorobis(1H-indazole)ruthenate (III)] is about 30 to about 70 weight percent of the composition,

mannitol is about 30 to about 70 weight percent of the composition,

citric acid is about 0.001 to about 1 weight percent of the composition,

and sodium citrate is about 0.0001 to about 1 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and Ru^{III}Cl₃(Hind)₂(H₂O);

wherein:

sodium trans-[tetrachlorobis(1H-indazole)ruthenate (III)] is about 49.86 weight percent of the composition,

mannitol is about 49.86 weight percent of the composition,

citric acid is about 0.187 weight percent of the composition,

sodium citrate is about 0.093 weight percentage of the composition,

and Ru^{III}Cl₃(Hind)₂(H₂O) is not more than 0.5 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and Ru^{III}Cl₃(Hind)₂(H₂O);

wherein:

sodium trans-[tetrachlorobis(1H-indazole)ruthenate (III)] is about 40 to about 60 weight percent of the composition,

mannitol is about 40 to about 60 weight percent of the composition,

citric acid is about 0.01 to about 0.5 weight percent of the composition,

sodium citrate is about 0.001 to about 0.25 weight percentage of the composition,

and Ru^{III}Cl₃(Hind)₂(H₂O) is about 0 to about 0.5 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), and cesium;

wherein:

sodium trans-[tetrachlorobis(1H-indazole)ruthenate (III)] is about 30 to about 70 weight percent of the composition,

mannitol is about 30 to about 70 weight percent of the composition,

citric acid is about 0.001 to about 1 weight percent of the composition,

sodium citrate is about 0.0001 to about 1 weight percentage of the composition,

Ru^{III}Cl₃(Hind)₂(H₂O) is not more than 0.5 weight percentage of the composition,

and cesium is not more than 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and cesium;

wherein:

sodium trans-[tetrachlorobis(1H-indazole)ruthenate (III)] is about 49.61 weight percent of the composition,

mannitol is about 49.86 weight percent of the composition,

citric acid is about 0.187 weight percent of the composition,

sodium citrate is about 0.093 weight percentage of the composition

and cesium is about 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and cesium;

wherein:

sodium trans-[tetrachlorobis(1H-indazole)ruthenate (III)] is about 40 to about 60 weight percent of the composition,

mannitol is about 40 to about 60 weight percent of the composition,

citric acid is about 0.01 to about 0.5 weight percent of the composition,

sodium citrate is about 0.001 to about 0.25 weight percentage of the composition,

and cesium is about 0.1 to about 0.5 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, and cesium;

wherein:

sodium trans-[tetrachlorobis(1H-indazole)ruthenate (III)] is about 30 to about 70 weight percent of the composition,

mannitol is about 30 to about 70 weight percent of the composition,

citric acid is about 0.001 to about 1 weight percent of the composition,

sodium citrate is about 0.0001 to about 1 weight percentage of the composition,

and cesium is about 0.01 to about 1 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:

sodium trans-[tetrachlorobis(1H-indazole)ruthenate (III)] about 46.61 weight percent of the composition,

mannitol is about 49.86 weight percent of the composition,

citric acid is about 0.187 weight percent of the composition,

sodium citrate is about 0.093 weight percentage of the composition,

Ru^{III}Cl₃(Hind)₂(H₂O) is not more than 0.5 weight percentage of the composition,

Ru^{III}Cl₃(Hind)₂(CH₃CN) is not more than 1.25 weight percentage of the composition,

Ru^{III}Cl₃(Hind)(HN═C(Me)ind) is not more than 1.0 weight percentage of the composition,

and cesium is not more than 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:

sodium trans-[tetrachlorobis(1H-indazole)ruthenate (III)] about between 46.61 weight percent of the composition,

mannitol is about 49.86 weight percent of the composition,

citric acid is about 0.187 weight percent of the composition,

sodium citrate is about 0.093 weight percentage of the composition,

Ru^{III}Cl₃(Hind)₂(H₂O) is not more than 0.5 weight percentage of the composition,

Ru^{III}Cl₃(Hind)₂(CH₃CN) is not more than 1.25 weight percentage of the composition,

Ru^{III}Cl₃(Hind)(HN═C(Me)ind) is not more than 1.0 weight percentage of the composition,

and cesium is not more than 0.25 weight percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:

sodium trans-[tetrachlorobis(1H-indazole)ruthenate (III)] is about 40 to about 60 weight percent of the composition,

mannitol is about 40 to about 60 weight percent of the composition,

citric acid is about 0.01 to about 0.5 weight percent of the composition,

sodium citrate is about 0.001 to about 0.25 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

Ru^{III}Cl₃(Hind)(HN═C(Me)ind) is not more than about 1.0 weight percentage of the composition,

and cesium is not more than 0.25 percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium;

wherein:

sodium trans-[tetrachlorobis(1H-indazole)ruthenate (III)] is about 30 to about 70 weight percent of the composition,

mannitol is about 30 to about 70 weight percent of the composition,

citric acid is about 0.001 to about 1 weight percent of the composition,

sodium citrate is about 0.0001 to about 1 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

Ru^{III}Cl₃(Hind)(HN═C(Me)ind) is not more than about 1.0 weight percentage of the composition,

and cesium is not more than 0.25 percentage of the composition. In some such embodiments, the composition is a lyophilized powder.

One embodiment of the present invention provides a composition comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium,

wherein:

sodium trans-[tetrachlorobis(1H-indazole)ruthenate (III)] is about 20 to about 80 weight percent of the composition,

mannitol is about 20 to about 80 weight percent of the composition,

citric acid is about 0.0001 to about 5 weight percent of the composition,

sodium citrate is about 0.00001 to about 5 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

Ru^{III}Cl₃(Hind)(HN═C(Me)ind) is not more than about 1.0 weight percentage of the composition, and cesium is not more than 0.25 percentage of the composition. In some such embodiments, the composition is a lyophilized powder.

In some embodiments, the present invention provides a unit dosage form comprising a formulation or composition described herein. The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism will depend upon a variety of factors including the disorder being treated and the severity of the disorder; activity of specific active agent employed; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

Compositions of the present invention can be provided as a unit dosage form. In some embodiments, a vial comprising sodium trans-[tetrachlorobis(1H-indazole)ruthenate (III)], mannitol, citric acid, sodium citrate is a unit dosage form.

In some embodiments, the present invention a vial comprising sodium trans-[tetrachlorobis(1H-indazole)ruthenate (III)], mannitol, citric acid, sodium citrate, and cesium is a unit dosage form.

In some embodiments, the present invention a vial comprising sodium trans-[tetrachlorobis(1H-indazole)ruthenate (III)], mannitol, citric acid, sodium citrate, Ru^{III}Cl₃(Hind)₂(H₂O), Ru^{III}Cl₃(Hind)₂(CH₃CN), Ru^{III}Cl₃(Hind)(HN═C(Me)ind), and cesium is a unit dosage form.

Still further encompassed by the invention are pharmaceutical packs and/or kits comprising compositions described herein, or a unit dosage form comprising a provided composition, and a container (e.g., a foil or plastic package, or other suitable container). Optionally instructions for use are additionally provided in such kits.

In some embodiments, the present invention can be provided as a unit dosage form. Indeed, a vial comprising sodium trans-[tetrachlorobis(1H-indazole)ruthenate (III)], mannitol, citric acid, sodium citrate is a unit dosage form depicted in Table 3

TABLE 3
Pharmaceutical Components

| | | Weight | Amount/ |
|---|---|---|---|
| Component | Function | % | vial |
| sodium trans-[tetrachlorobis | Active | 47.5 | 100 mg |
| (1H-indazole)ruthenate (III)] | | | |
| Mannitol | Cryoprotectant | 47.5 | 100 mg |
| Citric Acid | Buffer component | 3.37 | 7.1 mg |
| Sodium citrate | Buffer component | 1.63 | 3.4 mg |

In some embodiments, the pharmaceutical components described in Table 3 further comprise cesium;

wherein:

cesium is not more than 0.25 weight percentage of the composition.

In some embodiments, the pharmaceutical components described in Table 3 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:

cesium is not more than about 0.25 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

and Ru^{III}Cl₃(Hind)(HN═C(Me)ind) is not more than about 1.0 weight percentage of the composition.

In some embodiments, the pharmaceutical composition is selected from those in Table 4:

TABLE 4
Pharmaceutical Component Ranges

| | | Weight % | | |
|---|---|---|---|---|
| Component | Function | Range | Amount/vial | |
| sodium trans-[tetrachlorobis | Active | 42.75-52.25 | 90-110 | mg |
| (1H-indazole)ruthenate (III)] | | | | |
| Mannitol | Cryoprotectant | 42.75-52.25 | 90-110 | mg |
| Citric Acid | Buffer component | 3.033-3.707 | 6.39-7.81 | mg |
| Sodium citrate | Buffer component | 1.467-1.793 | 3.06-3.74 | mg |

In some embodiments, the pharmaceutical components described in Table 4 further comprise cesium;

wherein:

cesium is not more than 0.25 weight percentage of the composition.

In some embodiments, the pharmaceutical components described in Table 4 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:

cesium is not more than about 0.25 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

and Ru^{III}Cl₃(Hind)(HN═C(Me)ind) is not more than about 1.0 weight percentage of the composition.

In some embodiments, the present invention can be provided as a unit dosage form. Indeed, a vial comprising sodium trans-[tetrachlorobis(1H-indazole)ruthenate (III)], mannitol, citric acid, sodium citrate is a unit dosage form depicted in Table 5:

TABLE 5
Pharmaceutical Components

| | | Weight | |
|---|---|---|---|
| Component | Function | % | Amount/vial |
| sodium trans-[tetrachlorobis | Active | 49.86 | 300 mg |
| (1H-indazole)ruthenate (III)] | | | |
| Mannitol | Cryoprotectant | 49.86 | 300 mg |
| Citric Acid | Buffer component | 0.188 | 1.13 mg |
| Sodium citrate | Buffer component | 0.092 | 0.55 mg |

In some embodiments, the pharmaceutical components described in Table 5 further comprise cesium;

wherein:

cesium is not more than 0.25 weight percentage of the composition.

In some embodiments, the pharmaceutical components described in Table 5 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:

cesium is not more than about 0.25 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

and Ru^{III}Cl₃(Hind)(HN═C(Me)ind) is not more than about 1.0 weight percentage of the composition.

In some embodiments, the pharmaceutical composition is selected from those in Table 6:

TABLE 6
Pharmaceutical Components

| | | Weight % | | |
|---|---|---|---|---|
| Component | Function | Range | Amount/vial | |
| sodium trans-[tetrachlorobis | Active | 44.87-54.85 | 270-330 | mg |
| (1H-indazole)ruthenate (III)] | | | | |
| Mannitol | Cryoprotectant | 44.87-54.85 | 270-330 | mg |
| Citric Acid | Buffer component | 0.169-0.207 | 1.02-1.24 | mg |
| Sodium citrate | Buffer component | 0.0828-0.1012 | 0.495-0.605 | mg |

In some embodiments, the pharmaceutical components described in Table 6 further comprise cesium;

wherein:

cesium is not more than 0.25 weight percentage of the composition.

In some embodiments, the pharmaceutical components described in Table 6 further comprise cesium, Ru^{III}Cl₃(Hind)₂(H₂O), Ru^{III}Cl₃(Hind)₂(CH₃CN), and Ru^{III}Cl₃(Hind)(HN═C(Me)ind);

wherein:

cesium is not more than about 0.25 weight percentage of the composition,

Ru^{III}Cl₃(Hind)₂(H₂O) is not more than about 0.5 weight percentage of the composition,

Ru^{III}Cl₃(Hind)₂(CH₃CN) is not more than about 1.25 weight percentage of the composition,

and Ru^{III}Cl₃(Hind)(HN═C(Me)ind) is not more than about 1.0 weight percentage of the composition.

In some embodiments, the pharmaceutical components are as described in any of Tables 3-6, and further comprise cesium. In some embodiments, cesium is present in an amount of about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1.0 weight percentage of the composition.

In some embodiments, the present invention provides a method for treating cancer in a subject in need thereof comprising administering to the subject a provided composition of IT-139 described above and herein. In some such embodiments, the subject is a human patient.

In some embodiments, the present invention provides a method for treating cancer in a subject in need thereof comprising administering a provided composition of IT-139 described above and herein in combination with a chemotherapeutic agent.

In some embodiments, the present invention provides a method for treating cancer in a subject in need thereof comprising administering a provided composition of IT-139 described above and herein in combination with an immuno-oncology agent.

According to another embodiment, the present invention relates to a method of treating a cancer selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia, comprising administering IT-139, or a pharmaceutically acceptable composition thereof. IT-139 may for example be used in combination with a chemotherapeutic agent and/or an immuno-oncology agent.

Another embodiment provides a method for treating cancer by reducing the amount of GRP78 in cancer cells following administration of IT-139, alone or in combination with etoxomir.

According to another embodiment, the present invention provides a method for treating cancer by reducing the amount of GRP78 in cancer cells following administration of IT-139 in combination with a chemotherapy agent, wherein the administration of IT-139, or a pharmaceutically acceptable composition thereof, results in a reduction in the amount of GRP78 as compared to administration of the chemotherapy agent.

According to another embodiment, the present invention provides a method for treating cancer by reducing the amount of GRP78 in cancer cells following administration of IT-139 in combination with an immune-oncology agent, wherein the administration of IT-139, or a pharmaceutically acceptable composition thereof, results in a reduction in the amount of GRP78 as compared to administration of the immune-oncology agent alone.

The order of administration of therapeutics should be carefully considered. Without wishing to be bound to any particular theory, the mechanism of action and down-regulation of GRP78 dictates that any chemotherapeutic agent should be administered first, followed by IT-139 for maximum therapeutic benefit. As stated above, treatment with a range of chemotherapeutic agents results in an increase ER stress, which induces production of GRP78. This process is a cellular survival mechanism. Administration of IT-139 decreases the level of stress-induced GRP78, which removes a cellular survival pathway. The ultimate result is increased cancer cell death and increased anti-tumor effect.

According to one embodiment of the present invention provides a method for treating cancer in a patient in need thereof, comprising the steps of:

1) administering to the patient a chemotherapy agent;

2) subsequently administering IT-139, or a pharmaceutically acceptable composition thereof; to the patient; and

3) optionally repeating steps 1 and 2.

In certain embodiments, the IT-139, or a pharmaceutically acceptable composition thereof, is administered 1 day after the chemotherapy agent. In other embodiments, IT-139, or a pharmaceutically acceptable composition thereof, is administered to the patient 1 week after the chemotherapy agent. In yet other embodiments, IT-139 is administered to a patient between 1 and seven days after the chemotherapy agent.

In certain embodiments, the IT-139, or a pharmaceutically acceptable composition thereof, is administered simultaneously with the chemotherapy agent. In certain embodiments, the IT-139, or a pharmaceutically acceptable composition thereof, and the chemotherapy agent are administered within about 20-28 hours of each other, or within about 22-26 hours of each other, or within about 24 hours of each other.

In certain embodiments, the IT-139, or a pharmaceutically acceptable composition thereof, is administered before the chemotherapy agent. In certain embodiments, the IT-139, or a pharmaceutically acceptable composition thereof, is administered at least about 8-16 hours before the chemotherapy agent, or at least about 10-14 hours before the chemotherapy agent, or at least about 12 hours before the chemotherapy agent.

In certain embodiments, the IT-139, or a pharmaceutically acceptable composition thereof, is administered at least about 20-28 hours before the chemotherapy agent, or at least about 22-26 hours before the chemotherapy agent, or at least about 24 hours before the chemotherapy agent.

In certain embodiments, the IT-139, or a pharmaceutically acceptable composition thereof, is administered at least about 44-52 hours before the chemotherapy agent, or at least about 46-50 hours before the chemotherapy agent, or at least about 48 hours before the chemotherapy agent.

In certain embodiments, the IT-139, or a pharmaceutically acceptable composition thereof, is administered at least about 64-80 hours before the chemotherapy agent, or at least about 70-74 hours before the chemotherapy agent, or at least about 72 hours before the chemotherapy agent.

In certain embodiments, the IT-139, or a pharmaceutically acceptable composition thereof, is administered before the chemotherapy agent. In certain embodiments, the IT-139, or a pharmaceutically acceptable composition thereof, is administered at least about 8-16 hours after the chemotherapy agent, or at least about 10-14 hours after the chemotherapy agent, or at least about 12 hours after the chemotherapy agent.

In certain embodiments, the IT-139, or a pharmaceutically acceptable composition thereof, is administered at least about 20-28 hours after the chemotherapy agent, or at least about 22-26 hours after the chemotherapy agent, or at least about 24 hours after the chemotherapy agent.

In certain embodiments, the IT-139, or a pharmaceutically acceptable composition thereof, is administered at least about 44-52 hours after the chemotherapy agent, or at least about 46-50 hours after the chemotherapy agent, or at least about 48 hours after the chemotherapy agent.

In certain embodiments, the IT-139, or a pharmaceutically acceptable composition thereof, is administered at least about 64-80 hours after the chemotherapy agent, or at least about 70-74 hours after the chemotherapy agent, or at least about 72 hours after the chemotherapy agent.

In certain embodiments, the chemotherapeutic agent is selected from the group consisting of gemcitabine, nanoparticle albumin paclitaxel, paclitaxel, docetaxel, cabazitaxel, oxaliplatin, cisplatin, carboplatin, doxorubicin, daunorubicin, sorafenib, everolimus and vemurafenib. In certain embodiments, the chemotherapeutic agent is gemcitabine.

According to one embodiment of the present invention provides a method for treating pancreatic cancer in a patient in need thereof, comprising the steps of:

1) administering a gemcitabine and albumin nanoparticle paclitaxel;

2) subsequently administering IT-139, or a pharmaceutically acceptable composition thereof; and

3) optionally repeating steps 1 and 2.

In certain embodiments, the IT-139, or a pharmaceutically acceptable composition thereof, is administered simultaneously with gemcitabine. In certain embodiments, the IT-139, or a pharmaceutically acceptable composition thereof, and gemcitabine are administered within about 20-28 hours of each other, or within about 22-26 hours of each other, or within about 24 hours of each other.

In certain embodiments, the IT-139, or a pharmaceutically acceptable composition thereof, is administered before gemcitabine. In certain embodiments, the IT-139, or a pharmaceutically acceptable composition thereof, is administered at least about 8-16 hours before gemcitabine, or at least about 10-14 hours before gemcitabine, or at least about 12 hours before gemcitabine.

In certain embodiments, the IT-139, or a pharmaceutically acceptable composition thereof, is administered at least about 20-28 hours before gemcitabine, or at least about 22-26 hours before gemcitabine, or at least about 24 hours before gemcitabine.

In certain embodiments, the IT-139, or a pharmaceutically acceptable composition thereof, is administered at least about 44-52 hours before gemcitabine, or at least about 46-50 hours before gemcitabine, or at least about 48 hours before gemcitabine.

According to one embodiment of the present invention provides a method for treating cancer in a patient in need thereof, comprising administering IT-139, or a pharmaceutically acceptable composition thereof, in combination with an immuno-oncology agent. In certain embodiments, the immune-oncology agent is administered to the patient prior to the administration of IT-139, or a pharmaceutically acceptable composition thereof.

In certain embodiments, the IT-139, or a pharmaceutically acceptable composition thereof, is administered simultaneously with the immuno-oncology agent. In certain embodiments, the IT-139, or a pharmaceutically acceptable composition thereof, and the immuno-oncology agent are administered within about 20-28 hours of each other, or within about 22-26 hours of each other, or within about 24 hours of each other.

In certain embodiments, the IT-139, or a pharmaceutically acceptable composition thereof, is administered before the immuno-oncology agent. In certain embodiments, the IT-139, or a pharmaceutically acceptable composition thereof, is administered at least about 8-16 hours before the immuno-oncology agent, or at least about 10-14 hours before the immuno-oncology agent, or at least about 12 hours before the immuno-oncology agent.

In certain embodiments, the IT-139, or a pharmaceutically acceptable composition thereof, is administered at least about 20-28 hours before the immuno-oncology agent, or at least about 22-26 hours before the immuno-oncology agent, or at least about 24 hours before the immuno-oncology agent.

In certain embodiments, the IT-139, or a pharmaceutically acceptable composition thereof, is administered at least about 44-52 hours before the immuno-oncology agent, or at least about 46-50 hours before the immuno-oncology agent, or at least about 48 hours before the immuno-oncology agent.

In certain embodiments, the IT-139, or a pharmaceutically acceptable composition thereof, is administered at least about 64-80 hours before the immuno-oncology agent, or at least about 70-74 hours before the immuno-oncology agent, or at least about 72 hours before the immuno-oncology agent.

In certain embodiments, the IT-139, or a pharmaceutically acceptable composition thereof, is administered after the immuno-oncology agent. In certain embodiments, the IT-139, or a pharmaceutically acceptable composition thereof, is administered at least about 8-16 hours after the immuno-oncology agent, or at least about 10-14 hours after the immuno-oncology agent, or at least about 12 hours after the immuno-oncology agent.

In certain embodiments, the IT-139, or a pharmaceutically acceptable composition thereof, is administered at least about 20-28 hours after the immuno-oncology agent, or at least about 22-26 hours after the immuno-oncology agent, or at least about 24 hours after the immuno-oncology agent.

In certain embodiments, the IT-139, or a pharmaceutically acceptable composition thereof, is administered at least about 44-52 hours after the immuno-oncology agent, or at least about 46-50 hours after the immuno-oncology agent, or at least about 48 hours after the immuno-oncology agent.

In certain embodiments, the IT-139, or a pharmaceutically acceptable composition thereof, is administered at least about 64-80 hours after the immuno-oncology agent, or at least about 70-74 hours after the immuno-oncology agent, or at least about 72 hours after the immuno-oncology agent.

In certain embodiments, the immune-oncology agent is selected from the group consisting of cytokines, checkpoint inhibitors and antibodies other than PD-1 antibodies. In certain embodiments, the immune-oncology agent is selected from the group consisting of interferon, interleukin, PD-L1 antibodies, alemtuzumab, ipilimumab, ofatumumab, atezolizumab and rituximab.

According to one embodiment of the present invention provides a method for treating cancer in a patient in need thereof, comprising administering IT-139, or a pharmaceutically acceptable composition thereof, in combination with a PD-1 antibody. In certain embodiments, the PD-1 antibody is administered prior to the administration of the IT-139, or a pharmaceutically acceptable formulation thereof.

According to one embodiment of the present invention provides a method for treating cancer in a patient in need thereof, comprising administering IT-139, or a pharmaceutically acceptable composition thereof, in combination with a PD-L1 antibody. In certain embodiments, the PD-L1 antibody is administered prior to the administration of the IT-139, or a pharmaceutically acceptable formulation thereof.

According to one embodiment of the present invention provides a method for treating cancer in a patient in need thereof, comprising administering IT-139, or a pharmaceutically acceptable composition thereof, in combination with an immune-oncology agent other than a PD-1 antibody. In certain embodiments, the immune-oncology agent other than a PD-1 antibody is administered prior to the administration of the IT-139, or a pharmaceutically acceptable formulation thereof.

A titratable dosage may for example be adapted to allow a patient to take the medication in doses smaller than the unit dose, wherein a “unit dose” is defined as the maximum dose of medication that can be taken at any one time or within a specific dosage period. Titration of doses will allow different patients to incrementally increase the dose until they feel that the medication is efficacious, as not all patients will require the same dose to achieve the same benefits. A person with a larger build or faster metabolism may require larger doses to achieve the same effect as another with a smaller build or slower metabolism. Therefore, a titratable dosage has advantages over a standard dosage form.

In select embodiments, formulations may be adapted to be delivered in such a way as to target one or more of the following: sublingual, buccal, oral, rectal, nasal, parenteral and via the pulmonary system. Formulations may for example be in one or more of the following forms: gel, gel spray, tablet, liquid, capsule, by injection, or for vaporization.

Conventional pharmaceutical practice may be employed to provide suitable formulations or compositions to administer the formulations to subjects. Routes of administration may for example include, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets.

Methods well known in the art for making formulations are found in, for example, “Remington: The Science and Practice of Pharmacy” (21st edition), ed. David Troy, 2006, Lippincott Williams & Wilkins. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Pharmaceutical compositions of the present invention may be in any form which allows for the composition to be administered to a patient. For example, the composition may be in the form of a solid, liquid or gas (aerosol). Pharmaceutical composition of the invention are formulated so as to allow the active ingredients contained therein to be bioavailable upon administration of the composition to a patient. Compositions that will be administered to a patient may take the form of one or more dosage units, where for example, a tablet, capsule or cachet may be a single dosage unit, and a container of the compound in aerosol form may hold a plurality of dosage units.

Materials used in preparing the pharmaceutical compositions should be pharmaceutically pure and non-toxic in the amounts used. The inventive compositions may include one or more compounds (active ingredients) known for a particularly desirable effect. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes a formulation of the present invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid formulation for oral administration, the composition may be formulated into a powder, granule, compressed tablet, pill, capsule, cachet, chewing gum, wafer, lozenges, or the like form. Such a solid composition will typically contain one or more inert diluents or edible carriers. In addition, one or more of the following adjuvants may be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex; fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent. When the composition is in the form of a capsule, e.g., a gelatin capsule, it may contain, in addition to materials of the above type, a liquid carrier such as polyethylene glycol or a fatty oil.

The formulation may be in the form of a liquid, e.g., an elixir, syrup, solution, aqueous or oily emulsion or suspension, or even dry powders which may be reconstituted with water and/or other liquid media prior to use. The liquid may be for oral administration or for delivery by injection, as two examples. When intended for oral administration, compositions may contain, in addition to the present compounds, one or more of a sweetening agent, thickening agent, preservative (e.g., alkyl p-hydoxybenzoate), dye/colorant and flavor enhancer (flavorant). In a composition intended to be administered by injection, one or more of a surfactant, preservative (e.g., alkyl p-hydroxybenzoate), wetting agent, dispersing agent, suspending agent (e.g., sorbitol, glucose, or other sugar syrups), buffer, stabilizer and isotonic agent may be included. The emulsifying agent may be selected from lecithin or sorbitol monooleate.

The liquid pharmaceutical formulations of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The pharmaceutical formulation may be intended for topical administration, in which case the carrier may suitably comprise a solution, emulsion, ointment, cream or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The formulation may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes may be melted, and the aminocyclohexyl ether compound is dispersed homogeneously therein by stirring. The molten homogeneous mixture is then poured into convenient sized molds, allowed to cool and thereby solidify.

The formulation may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule or cachet.

The pharmaceutical formulation may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit.

Some biologically active compounds may be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

The present invention also provides kits that contain a pharmaceutical formulation, together with instructions for the use of the formulation. Preferably, a commercial package will contain one or more unit doses of the formulation. Formulations which are light and/or air sensitive may require special packaging and/or formulation. For example, packaging may be used which is opaque to light, and/or sealed from contact with ambient air, and/or formulated with suitable coatings or excipients.

The formulations of the invention can be provided alone or in combination with other compounds (for example, small molecules, nucleic acid molecules, peptides, or peptide analogues), in the presence of a carrier or any pharmaceutically or biologically acceptable carrier. As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration. Pharmaceutically acceptable carriers generally include sterile aqueous solutions or dispersions and sterile powders. Supplementary active compounds can also be incorporated into the formulations.

An “effective amount” of a formulation according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a formulation may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the formulation or active compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. For any particular subject, the timing and dose of treatments may be adjusted over time (e.g., timing may be daily, every other day, weekly, monthly) according to the individual need and the professional judgment of the person administering or supervising the administration of the compositions.

In therapeutic applications, synergy between active ingredients occurs when an observed combined therapeutic effect is greater than the sum of therapeutic effects of individual active ingredients, or a new therapeutic effect is produced that the active ingredients could not produce alone. Accordingly, when components of a formulation are present in synergistically effective amounts, the formulation yields a therapeutic effect that is greater than would be achieved by the individual active ingredients administered alone at comparable dosages. In this context, the enhancement of therapeutic effect may take the form of increased efficacy or potency and/or decreased adverse effects. The synergistic effect may be mediated in whole or in part by the pharmacokinetics and/or pharmacodynamics of the active ingredients in a subject, so that the amount and proportion of the ingredients in the formulation may be synergistic in vivo. This in vivo synergy may be effected with a formulation that includes the active ingredients in amounts and proportions that are also synergistic in in vitro assays of efficacy. As used herein, the term “synergistically effective amounts” accordingly refers to amounts that are synergistic in vivo and/or in vitro. A numeric quantification of synergy is often expressed as a fractional inhibitory concentration index (FICI), which represents the sum of the fractional inhibitory concentrations (FICs) of each drug tested, where the FIC is determined for each drug by dividing the minimum inhibitory concentration (MIC, the lowest concentration of the drug which prevents visible growth of the bacterium in a standard in vitro assay—standard colorometric assay based on resazurin) of each drug when used in combination by the MIC of each drug when used alone. In very general terms, a FICI lower or higher than 1 indicates positively correlated activity (at least additive synergy) or an absence of positive interactions, respectively. More definitively, synergy of two compounds may be conservatively defined as a FICI of ≤0.5 (see Odds, 2003; with additivity or additive synergy corresponding to a FICI of >0.5 to ≤1; no interaction (indifference) corresponding to a FICI of >1 to ≤4; and antagonism corresponding to a FICI of >>4). Synergy of three compounds has been defined as a FICI of ≤1.0. (Berenbaum, 1978; Yu et al., 1980).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Terms such as “exemplary” or “exemplified” are used herein to mean “serving as an example, instance, or illustration.” Any implementation described herein as “exemplary” or “exemplified” is accordingly not to be construed as necessarily preferred or advantageous over other implementations, all such implementations being independent embodiments. Unless otherwise stated, numeric ranges are inclusive of the numbers defining the range, and numbers are necessarily approximations to the given decimal. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification, and all documents cited in such documents and publications, are hereby incorporated herein by reference as if each individual publication were specifically and individually indicated to be incorporated by reference herein and as though fully set forth herein. The invention includes all embodiments and variations substantially as hereinbefore described and with reference to the examples and drawings.

In some embodiments, the invention excludes steps that involve medical or surgical treatment.

1. A method for treating a cancer in a human patient in need thereof, comprising administering an effective amount of sodium trans-[tetrachloridobis(1H-indazole)ruthenate(III)] and an effective amount of etomoxir.

2. The method of claim 1, wherein the effective amount of sodium trans-[tetrachloridobis(1H-indazole)ruthenate(III)] and the effective amount of etomoxir are synergistically effective for treating the cancer.

3. The method of claim 1 or 2, wherein the cancer is a cancer that is resistant to treatment with sodium trans-[tetrachloridobis(1H-indazole)ruthenate(III)] alone.

4. The method according to any one of claims 1 to 3, wherein the cancer is a colon cancer or a pancreatic cancer.

5. The method according to any one of claims 1 to 4, wherein the sodium trans-[tetrachloridobis(1H-indazole)ruthenate(III)] and the etomoxir are for sequential administration, in any order, or are for combined administration in a co-formulation.

6. Use of sodium trans-[tetrachloridobis(1H-indazole)ruthenate(III)] in combination with etomoxir for treating a cancer in a human patient.

7. The use according to claim 6, wherein the sodium trans-[tetrachloridobis(1H-indazole)ruthenate(III)] and the etomoxir are for use in synergistically effective amounts for treating the cancer.

8. The use according to claim 6 or 7, wherein the cancer is a cancer that is resistant to treatment with sodium trans-[tetrachloridobis(1H-indazole)ruthenate(III)] alone.

9. The use according to any one of claims 6 to 8, wherein the cancer is a colon cancer or a pancreatic cancer.

10. The use according to any one of claims 6 to 9, wherein the sodium trans-[tetrachloridobis(1H-indazole)ruthenate(III)] and to etomoxir are for sequential use, or are for combined use in a co-formulation.
